(19) United States
(12) Patent Application Publication
(10) Pub. No.: US 2019/0156916 A1
(43) Pub. Date: May 23, 2019

(54) Data Processing Method and Apparatus, and Computing Node

(71) Applicant: Huawei Technologies Co., Ltd., Shenzhen (CN)

(72) Inventors: Liqun Deng, Shenzhen (CN); Guowei Huang, Shenzhen (CN); Jiansheng Wei, Shenzhen (CN)

(21) Appl. No.: 16/251,829

(22) Filed: Jan. 18, 2019

Related U.S. Application Data
(63) Continuation of application No. PCT/CN2016/099739, filed on Sep. 22, 2016.

(51) Int. Cl. G16B 30/10; G06F 17/27; G06F 9/48; G06F 9/38

(52) U.S. Cl. CPC G16B 30/10; G06F 17/2705; G06F 9/3855; G06F 9/3877; G06F 9/48

(57) ABSTRACT

A data processing method includes distributing, by a computing node, a pasting back result sequence corresponding to a to-be-pasted-back deoxyribonucleic acid (DNA) read string to a pasting back result sequence set corresponding to a target chromosome region, when the quantity of the pasting back result sequences included in the pasting back result sequence set is greater than or equal to the pre-determined quantity threshold, dividing the pasting back result sequence set into k pasting back result sequence subsets according to a preset division rule, and dividing the target chromosome region into k chromosome subregions in a one-to-one correspondence to the k pasting back result sequence subsets, and further dividing a gene analysis task of the pasting back result sequence set into k gene analysis subtasks, and executing in parallel the k gene analysis subtasks.

### CROSS-REFERENCE TO RELATED APPLICATIONS

This application is a continuation of International Patent Application No. PCT/CN2016/099739 filed on Sep. 22, 2016, which is hereby incorporated by reference in its entirety.

### TECHNICAL FIELD

The present disclosure relates to the field of gene analysis technologies, and in particular, to a data processing method and apparatus, and a computing node.

### BACKGROUND

With development of a deoxyribonucleic acid (DNA) sequencing technology, gene analysis has become an important means for detection and targeted treatment for hereditary and mutation diseases. Generally, the gene analysis includes three phases, DNA sequencing, DNA sequence assembly and mutation identification, and gene annotation and analysis. The DNA sequence assembly and mutation identification require a large quantity of computing overheads, and an entire gene analysis task process is extremely time-consuming. Currently, it has been proposed to use a parallel computing framework, such as HADOOP/SPARK to establish an extensible genome analysis task pipeline. A gene analysis task is divided, according to a data dimension, into a plurality of tasks for parallel execution in a computer cluster in order to reduce time overheads of the gene analysis task. However, in practice, because of many possible factors, such as different sequencing depths of the DNA sequencing in each chromosome region and uneven distribution of processed results of sequencing data obtained after several steps, an uneven data problem may occur in a small quantity of tasks, that is, a data amount processed in an uneven data task is far greater than an average data amount required to be processed in another task. This further causes a serious long tail problem, that is, an execution time of the uneven data task is far greater than an execution time of another task, thereby affecting execution efficiency of the entire genome analysis task pipeline.

To resolve the foregoing uneven data problem, existing solutions include Solution 1: A data balancing module is increased, and the data balancing module divides an uneven data group into two data sub-groups such that each data group and each data sub-group respectively correspond to a gene analysis task, and these gene analysis tasks are executed in parallel in the computer cluster, Solution 2: A relatively large quantity of computing resources are allocated for an uneven data task, and Solution 3: An uneven data task is dynamically divided into a plurality of tasks, and the plurality of tasks are allocated to a computing node having idle computing resources for execution. Solution 1 is not applicable to a large-scale DNA data processing scenario. In Solution 2, optimal computing resources required by a gene analysis task in each phase are different, and therefore an increase of the allocated computing resources cannot always reduce an execution time of the gene analysis task. In Solution 3, it is hard to execute the gene analysis task in practice. In the gene analysis task, a key-value set required to be processed in each task includes only a single key (a key is usually a chromosome subregion) in most cases, such a data set cannot be dynamically divided in a task running process. It can be learned that how to improve execution efficiency of the gene analysis task and reduce the execution time of the gene analysis task has become an urgent problem that needs to be resolved.

### SUMMARY

Embodiments of the present disclosure disclose a data processing method and apparatus, and a computing node in order to improve execution efficiency of a gene analysis task and reduce time overheads of the gene analysis task.

According to a first aspect of the embodiments of the present disclosure, a data processing method is disclosed, where the method is applied to a distributed computing system, the system includes a plurality of computing nodes, and the method includes performing a pasting back operation, by a first computing node by aligning a to-be-pasted-back DNA read string with a reference gene sequence, and obtaining a chromosome location matching the to-be-pasted-back DNA read string, determining a target chromosome region in which the chromosome location is located, and distributing a pasting back result sequence that is obtained after the pasting back operation is performed and that is corresponding to the to-be-pasted-back DNA read string to a pasting back result sequence set corresponding to the target chromosome region, where all pasting back result sequences corresponding to one chromosome region are collectively referred to as a pasting back result sequence set, and the first computing node is any one of the plurality of computing nodes, determining, by the first computing node, whether a quantity of pasting back result sequences included in the pasting back result sequence set is greater than or equal to a pre-determined quantity threshold, and determining that a gene analysis task for the target chromosome region is an uneven task, dividing the pasting back result sequence set into k pasting back result sequence subsets according to a preset division rule, and correspondingly dividing the target chromosome region into k chromosome subregions if the quantity of the pasting back result sequences included in the pasting back result sequence set is greater than or equal to the pre-determined quantity threshold, where the k chromosome subregions are in a one-to-one correspondence to the k pasting back result sequence subsets, and k is an integer greater than or equal to 2, and dividing, by the first computing node, a gene analysis task of the pasting back result sequence set corresponding to the target chromosome region into k gene analysis subtasks, and executing in parallel the k gene analysis subtasks using a computing resource allocated by the distributed computing system to the first computing node in order to complete the gene analysis task of the pasting back result sequence set corresponding to the target chromosome region, thereby improving execution efficiency of the gene analysis task and reduce time overheads of the gene analysis task.

Optionally, before determining, by the first computing node, whether a quantity of pasting back result sequences included in the pasting back result sequence set is greater than or equal to a pre-determined quantity threshold, the quantity threshold is first calculated, and a calculation manner may be as follows. Obtaining, by the first computing node, a size of a data amount of all to-be-pasted-back DNA read strings, determining, according to the size of the data amount of all the to-be-pasted-back DNA read strings, a size of a data amount of a pasting back result sequence obtained after all the to-be-pasted-back DNA read strings are pasted back to the reference gene sequence, determining, according to a quantity of the plurality of chromosome regions obtained through division in advance and the size of the data amount of the pasting back result sequence, a size of an average data amount of a pasting back result sequence set corresponding to each chromosome region, and determining the quantity threshold with reference to a quantity of pasting back result sequences in a unit of data amount, where the quantity threshold is used as a criterion for determining whether a gene analysis task for a chromosome region is an uneven task.

Optionally, a specific step in which the first computing node divides the pasting back result sequence set into k pasting back result sequence subsets according to the preset division rule, and correspondingly divides the target chromosome region into k chromosome subregions may be as follows.

The first computing node determines, according to a ratio of the quantity of the pasting back result sequences included in the pasting back result sequence set to the pre-determined quantity threshold, the quantity of the pasting back result sequence subsets k into which the pasting back result sequence set requires to be divided, where for example, k is a result obtained after a rounding operation is performed on the ratio. The first computing node divides the pasting back result sequence set into k pasting back result sequence subsets, and correspondingly divides the target chromosome region into k consecutive chromosome subregions, and further distributes, according to a chromosome subregion in which a chromosome location corresponding to each pasting back result sequence included in the pasting back result sequence set is located, each pasting back result sequence included in the pasting back result sequence set to the k pasting back result sequence subsets corresponding to the k consecutive chromosome subregions, where one pasting back result sequence subset is data that needs to be processed in one gene analysis subtask.

Optionally, the method further includes that if a chromosome location corresponding to the target pasting back result sequence in the pasting back result sequence set is located in two chromosome subregions, the first computing node may distribute the target pasting back result sequence to pasting back result sequence subsets corresponding to both the two chromosome subregions in order to ensure that all data corresponding to the target pasting back result sequence may be processed, and further ensure integrality of a result of the gene analysis task.

Optionally, after executing in parallel the k gene analysis subtasks, the method further includes combining, by the first computing node, results of the k gene analysis subtasks, and using a combined result as a result of the gene analysis task of the pasting back result sequence set corresponding to the target chromosome region.

Optionally, the gene analysis task further includes one or more of deduplication, local reordering, base quality recalibration, or mutation detection.

According to a second aspect of the embodiments of the present disclosure, a data processing apparatus is disclosed, where the apparatus is applied to a distributed computing system, and the apparatus includes an obtaining module configured to perform a pasting back operation by aligning a to-be-pasted-back DNA read string with a reference gene sequence, and obtain a chromosome location matching the to-be-pasted-back DNA read string, a determining module configured to determine, from a plurality of chromosome regions obtained through division in advance, a target chromosome region in which the chromosome location is located, a distribution module configured to distribute a pasting back result sequence that is obtained after the pasting back operation is performed and that is corresponding to the to-be-pasted-back DNA read string to a pasting back result sequence set corresponding to the target chromosome region, where all pasting back result sequences corresponding to one chromosome region are collectively referred to as a pasting back result sequence set, a judging module configured to determine whether a quantity of pasting back result sequences included in the pasting back result sequence set is greater than or equal to a pre-determined quantity threshold, where the distribution module is further configured to determine that a gene analysis task for the target chromosome region is an uneven task, divide the pasting back result sequence set into k pasting back result sequence subsets according to a preset division rule, and correspondingly divide the target chromosome region into k chromosome subregions when the judging module determines that the quantity of the pasting back result sequences included in the pasting back result sequence set is greater than or equal to the pre-determined quantity threshold, where the k chromosome subregions are in a one-to-one correspondence to the k pasting back result sequence subsets, and k is an integer greater than or equal to 2, and the distribution module is further configured to divide the gene analysis task of the pasting back result sequence set corresponding to the target chromosome region into k gene analysis subtasks, where the k gene analysis subtasks are in a one-to-one correspondence to the k chromosome subregions, and an execution module configured to execute in parallel the k gene analysis subtasks using a computing resource allocated by the distributed computing system in order to complete the gene analysis task of the pasting back result sequence set corresponding to the target chromosome region, thereby improving the execution efficiency of the gene analysis task and reduce the time overheads of the gene analysis task.

Optionally, the obtaining module is further configured to obtain a size of a data amount of all to-be-pasted-back DNA read strings.

The determining module is further configured to determine, according to the size of the data amount of all the to-be-pasted-back DNA read strings, a size of a data amount of a pasting back result sequence obtained after all the to-be-pasted-back DNA read strings are pasted back to the reference gene sequence.

The determining module is further configured to determine, according to a quantity of the plurality of chromosome regions obtained through division in advance and the size of the data amount of the pasting back result sequence, a size of an average data amount of a pasting back result sequence set corresponding to each chromosome region.

The determining module is further configured to determine the pre-determined quantity threshold according to the average data amount of the pasting back result sequence set corresponding to each chromosome region and a quantity of pasting back result sequences in a unit of data amount, where the quantity threshold is used as a criterion for determining whether a gene analysis task for a chromosome region is an uneven task.

Optionally, the distribution module may further include a determining unit configured to determine, according to a ratio of the quantity of the pasting back result sequences included in the pasting back result sequence set to the pre-determined quantity threshold, a quantity of the pasting back result sequence subsets k obtained after the pasting back result sequence set is divided, and a distribution unit configured to divide the pasting back result sequence set into k pasting back result sequence subsets, and divide the target chromosome region into k consecutive chromosome subregions, where the distribution unit is further configured to correspondingly distribute, according to a chromosome subregion in which a chromosome location corresponding to each pasting back result sequence included in the pasting back result sequence set is located, each pasting back result sequence included in the pasting back result sequence set to the k pasting back result sequence subsets corresponding to the k consecutive chromosome subregions, where one pasting back result sequence subset is data that needs to be processed in one gene analysis subtask.

Optionally, when a chromosome location corresponding to a target pasting back result sequence in the pasting back result sequence set is located in two chromosome subregions, the distribution unit is further configured to distribute the target pasting back result sequence to pasting back result sequence subsets corresponding to both the two chromosome subregions in order to ensure that all data corresponding to the target pasting back result sequence may be processed, and further ensure integrality of a result of the gene analysis task.

Optionally, the apparatus further includes a combination module configured to combine results of the k gene analysis subtasks, and use a combined result as a result of the gene analysis task of the pasting back result sequence set corresponding to the target chromosome region after the execution module executes in parallel the k gene analysis subtasks.

Optionally, the gene analysis task further includes one or more of deduplication, local reordering, base quality recalibration, or mutation detection.

According to a third aspect of the embodiments of the present disclosure, a computing node is disclosed, where the computing node is applied to a distributed computing system, the computing node includes a processor and a memory, the processor and the memory are connected using a bus, the memory stores executable program code, and the processor is configured to invoke the executable program code to perform the data processing method according to any one of the foregoing first aspect.

In the embodiments of the present disclosure, the computing node distributes the pasting back result sequence corresponding to the to-be-pasted-back DNA read string to the pasting back result sequence set corresponding to the target chromosome region, determines whether the quantity of the pasting back result sequences included in the pasting back result sequence set is greater than or equal to the pre-determined quantity threshold, and if the quantity of the pasting back result sequences included in the pasting back result sequence set is greater than or equal to the pre-determined quantity threshold, divides the pasting back result sequence set into k pasting back result sequence subsets according to the preset division rule, and correspondingly divides the target chromosome region into k chromosome subregions in a one-to-one correspondence to the k pasting back result sequence subsets, and further divides the gene analysis task of the pasting back result sequence set into k gene analysis subtasks, and executes in parallel the k gene analysis subtasks. This can improve execution efficiency of the gene analysis task and reduce time overheads of the gene analysis task.

### BRIEF DESCRIPTION OF DRAWINGS

To describe the technical solutions in some of the embodiments of the present disclosure more clearly, the following briefly describes the accompanying drawings describing some of the embodiments. The accompanying drawings in the following description show merely some embodiments of the present disclosure, and a person of ordinary skill in the art may still derive other drawings from these accompanying drawings without creative efforts.

FIG. 1 is a schematic flowchart of a gene analysis task according to an embodiment of the present disclosure;

FIG. 2A and FIG. 2B are a schematic flowchart of a data processing method according to an embodiment of the present disclosure;

FIG. 3 is a schematic structural diagram of a data processing apparatus according to an embodiment of the present disclosure; and

FIG. 4 is a schematic structural diagram of a computing node according to an embodiment of the present disclosure.

### DESCRIPTION OF EMBODIMENTS

The following clearly describes the technical solutions in the embodiments of the present disclosure with reference to the accompanying drawings in the embodiments of the present disclosure. The described embodiments are merely a part rather than all of the embodiments of the present disclosure. All other embodiments obtained by a person of ordinary skill in the art based on the embodiments of the present disclosure without creative efforts shall fall within the protection scope of the present disclosure.

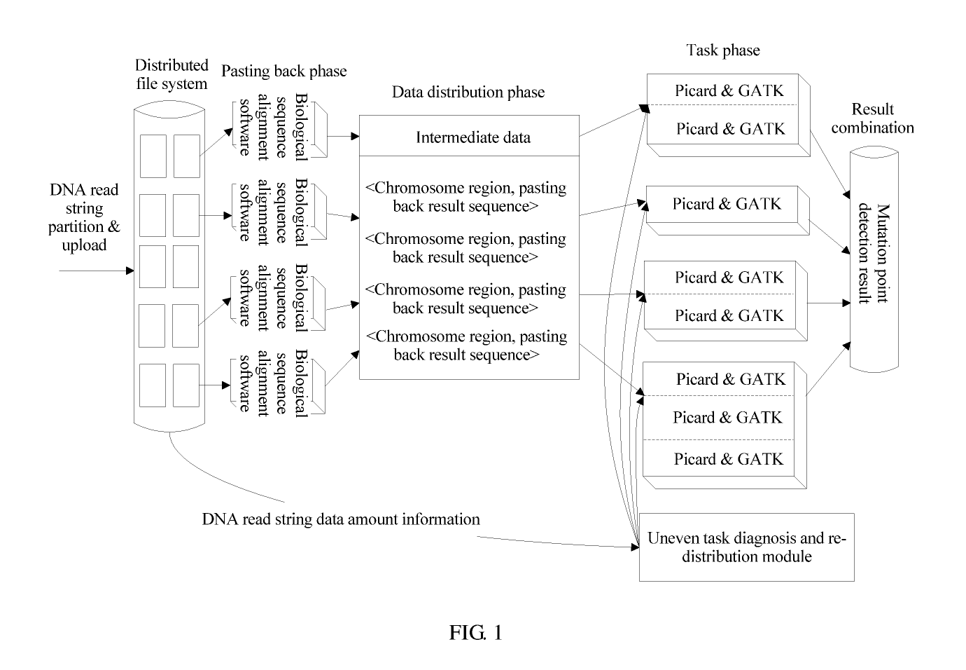

Referring to FIG. 1, FIG. 1 is a schematic flowchart of a gene analysis task according to an embodiment of the present disclosure. When an existing distributed computing system HADOOP MapReduce model is used as an example, the gene analysis task described in this embodiment includes the following procedures.

1) DNA of a biospecimen is extracted using a device such as a DNA sequencer, and the DNA is converted into a DNA read string that can be identified by a computer. Each DNA read string represents a fixed-length character string that can be identified by a computer and that includes four characters, A (which represents adenine), T (which represents thymine), C (which represents cytosine), and G (which represents guanine). The DNA read string may be usually stored in a file in a format such as FASTQ or FASTA.

2) The DNA read string that is output by the sequencer is divided into a plurality of data blocks, and the plurality of data blocks are stored in a distributed file system (e.g., HADOOP Distributed File System (HDFS)).

3) A pasting back operation is performed in a Map phase, that is, a biological sequence alignment software tool (for example, Burrows-Wheeler Aligner (BWA) software) is used to paste the DNA read string back to a reference gene sequence in order to determine a chromosome location matching each DNA read string, and obtain a corresponding pasting back result sequence, where the pasting back result sequence may be usually referred to as a sequence alignment map (SAM) record, and a quantity of Map tasks is equal to a quantity of the data blocks into which the DNA read string is divided in 2).

4) A data distribution phase: All pasting back result sequences corresponding to the pasting back operation are distributed to corresponding Reduce tasks according to chromosome regions to which the pasting back result sequences are pasted back, where one Reduce task is a gene analysis task for one chromosome region.

5) A Reduce phase: Steps such as deduplication, local reordering, base quality recalibration, and mutation detection are sequentially performed using Picard and GATK.

A newly added uneven task diagnosis and re-distribution module may be a software program that runs on a computing node or all computing nodes of the distributed computing system. The uneven task is a task in which a processed data amount is far greater than an average data amount required to be processed in other tasks such that an execution time of the task is far longer than an execution time of other tasks.

In this embodiment of the present disclosure, before the foregoing step 5), the uneven task diagnosis and re-distribution module may determine, according to information such as a data amount of the DNA read string, whether each Reduce task is an uneven task. An uneven Reduce task is locally re-divided on a computing node, that is, the uneven Reduce task is divided into two, three, or more Reduce subtasks (as shown in FIG. 1), and a computing resource allocated by the distributed computing system to the uneven Reduce task is used to execute in parallel the Reduce subtasks obtained through division, and finally results of all the Reduce subtasks are combined, and a mutation point detection result is output. This can improve execution efficiency of the gene analysis task and reduce time overheads of the gene analysis task.

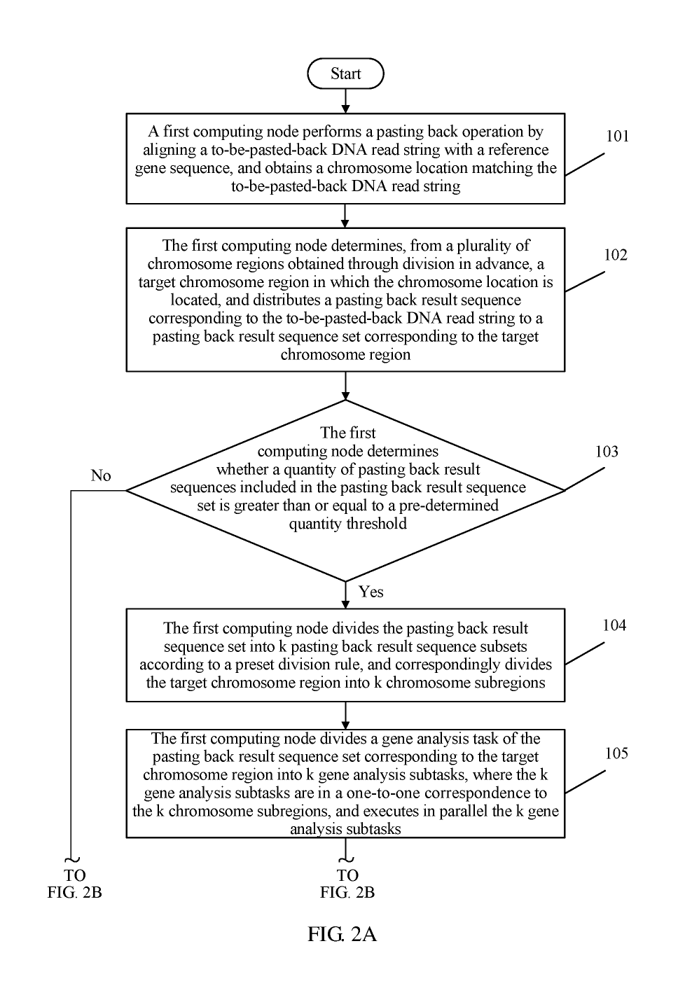

Referring to FIG. 2A and FIG. 2B, FIG. 2A and FIG. 2B are a schematic flowchart of a data processing method according to an embodiment of the present disclosure. The data processing method described in this embodiment is applied to a distributed computing system, where the system includes a plurality of computing nodes. The method includes the following steps.

Step 101. A first computing node performs a pasting back operation by aligning a to-be-pasted-back DNA read string with a reference gene sequence, and obtains a chromosome location matching the to-be-pasted-back DNA read string.

The first computing node is any one of the plurality of computing nodes. The pasting back operation means that the first computing node aligns the to-be-pasted-back DNA read string with the reference gene sequence to obtain the chromosome location matching the to-be-pasted-back DNA read string, obtains a pasting back result sequence corresponding to the to-be-pasted-back DNA read string at the same time, and converts the pasting back result sequence into a key-value pair output like <chromosome region, pasting back result sequence>.

Step 102. The first computing node determines, from a plurality of chromosome regions obtained through division in advance, a target chromosome region in which the chromosome location is located, and distributes a pasting back result sequence corresponding to the to-be-pasted-back DNA read string to a pasting back result sequence set corresponding to the target chromosome region.

All pasting back result sequences belonging to a same chromosome region need to be allocated to a same gene analysis task (that is, the foregoing Reduce task).

Further, a chromosome of a biospecimen is divided into a plurality of chromosome regions in advance. The first computing node may determine, according to the chromosome location matching the to-be-pasted-back DNA read string, a target chromosome region to which the pasting back result sequence corresponding to the to-be-pasted-back DNA read string belongs, and distribute the pasting back result sequence to a pasting back result sequence set corresponding to the target chromosome region. That is, all pasting back result sequences included in the pasting back result sequence set corresponding to the target chromosome region may be allocated to a same gene analysis task.

Step 103. The first computing node determines whether a quantity of pasting back result sequences included in the pasting back result sequence set is greater than or equal to a pre-determined quantity threshold, and if the quantity of the pasting back result sequences included in the pasting back result sequence set is greater than or equal to the pre-determined quantity threshold, perform steps 104-106, or if the quantity of the pasting back result sequences included in the pasting back result sequence set is smaller than the pre-determined quantity threshold, perform step 107.

During specific implementation, after all DNA read strings corresponding to DNA of the biospecimen are pasted back, the first computing node determines whether the quantity of the pasting back result sequences included in the pasting back result sequence set processed by the first computing node is greater than or equal to the pre-determined quantity threshold.

The pre-determined quantity threshold may be an average value of a quantity of pasting back result sequences included in a pasting back result sequence set corresponding to each chromosome region.

In some feasible implementations, a process of determining the pre-determined quantity threshold may be obtain a size of a data amount of all to-be-pasted-back DNA read strings, determine, according to the size of the data amount of all the to-be-pasted-back DNA read strings, a size of a data amount of a pasting back result sequence obtained after all the to-be-pasted-back DNA read strings are pasted back to the reference gene sequence, determine, according to a quantity of the plurality of chromosome regions obtained through division in advance and the size of the data amount of the pasting back result sequence, a size of an average data amount of a pasting back result sequence set corresponding to each chromosome region, and determine the pre-determined quantity threshold according to the average data amount of the pasting back result sequence set corresponding to each chromosome region and a quantity of pasting back result sequences in a unit of data amount.

It should be noted that the pre-determined quantity threshold may be determined before step 103 or may be first determined before step 101. This is not limited in this embodiment of the present disclosure.

For example, if the pre-determined quantity threshold is first determined before step 101, the size of the data amount of all the to-be-pasted-back DNA read strings may be obtained, which is assumed to be M.

The size of the data amount of the pasting back result sequence obtained after all the to-be-pasted-back DNA read strings are pasted back is estimated. Based on an implementation result, the pasting back result sequence is linearly proportional to the DNA read string in a size of a data amount. It is assumed that the size of the data amount of the pasting back result sequence is S. S=πM, where π is a proportionality coefficient and is related to a software tool type used in a pasting back operation. If BWA software is used, a value of π may be 4.42.

The size of the average data amount of the pasting back result sequence set corresponding to each chromosome region is calculated S_{avg}=S/R, where R is a quantity of chromosome regions.

The pre-determined quantity threshold is determined Λ=λS_{avg}, where λ is a quantity of pasting back result sequences in a unit of data amount (for example, 1 gigabyte (GB)).

Certainly, if the pre-determined quantity threshold is determined before step 103, the size of the data amount of the pasting back result sequence obtained after all the to-be-pasted-back DNA read strings are pasted back may be directly read without estimation, and the obtained pre-determined quantity threshold may be more accurate.

It should be noted that the pre-determined quantity threshold may be determined by only one (for example, the first computing node) of computing nodes. The first computing node may notify other computing nodes of the determined pre-determined quantity threshold in a broadcast manner and the like. Alternatively, each computing node may determine the pre-determined quantity threshold using the foregoing method.

Step 104. The first computing node divides the pasting back result sequence set into k pasting back result sequence subsets according to a preset division rule, and correspondingly divides the target chromosome region into k chromosome subregions, where the k chromosome subregions are in a one-to-one correspondence to the k pasting back result sequence subsets, and k is an integer greater than or equal to 2.

In the distributed computing system, if the first computing node determines that the quantity of the pasting back result sequences included in the pasting back result sequence set processed by the first computing node is greater than or equal to the pre-determined quantity threshold, the first computing node determines that a gene analysis task corresponding to the pasting back result sequence set processed by the first computing node is an uneven task and needs to be locally divided according to the preset division rule.

Further, the preset division rule may be that the first computing node determines, according to a ratio of the quantity of the pasting back result sequences N included in the pasting back result sequence set to the pre-determined quantity threshold A, a quantity of the pasting back result sequence subsets k obtained after the pasting back result sequence set is divided. For example, k is a result obtained after a rounding operation is performed on the ratio, that is, k=[N/A], where [ ] indicates the rounding operation. The first computing node divides the pasting back result sequence set into k pasting back result sequence subsets, divides the target chromosome region into k consecutive chromosome subregions, and then may correspondingly distribute, according to a chromosome subregion in which a chromosome location corresponding to each pasting back result sequence included in the pasting back result sequence set is located, each pasting back result sequence included in the pasting back result sequence set to the k pasting back result sequence subsets corresponding to the k consecutive chromosome subregions.

For example, input data of the gene analysis task of the pasting back result sequence set corresponding to the target chromosome region is D=<the target chromosome region Rr, List (pasting back result sequence)>. The first computing node calculates an average value of a quantity of pasting back result sequences included in each pasting back result sequence subset in the k pasting back result sequence subset n=[N/k], where H indicates the rounding operation in order to ensure as much as possible that the pasting back result sequence set is divided into k pasting back result sequence subsets, each of which includes a same quantity of pasting back result sequences. The first computing node divides the target chromosome region into k consecutive chromosome subregions Rr₁, Rr₂, . . . , Rrₖ. It is assumed that an interval range of the target chromosome region Rr in the chromosome of the biospecimen is [x, y].

An interval of Rr₁ is [x₁, y₁], where x₁=x, and y₁=a start coordinate of a chromosome corresponding to an (n+1)^{th} pasting back result sequence in D.

An interval of Rr₁ is [xᵢ, yᵢ], where xᵢ=yᵢ₋₁+1, yᵢ=a start coordinate of a chromosome corresponding to a (i×n+1)^{th} pasting back result sequence in D, and 1<i<k.

It should be noted that the average value n of the quantity of the pasting back result sequences included in each pasting back result sequence subset is obtained by performing the rounding operation. Therefore, if a quantity of pasting back result sequences N included in the pasting back result sequence set is greater than n×k, a part exceeding n×k in the pasting back result sequence set may be distributed to a k^{th} chromosome subregion Rrₖ.

An interval of Rrₖ is [xₖ, yₖ], where xₖ=yₖ₋₁+1, and yₖ=y.

Further, if a chromosome location corresponding to a target pasting back result sequence in the pasting back result sequence set is completely located in one chromosome subregion, the first computing node distributes the target pasting back result sequence to a pasting back result sequence subset corresponding to the chromosome subregion. If a coordinate range of the chromosome location corresponding to the target pasting back result sequence in the pasting back result sequence set is relatively large and is located in two chromosome subregions, the first computing node distributes the target pasting back result sequence to pasting back result sequence subsets corresponding to both the two chromosome subregions in order to ensure that all data corresponding to the target pasting back result sequence may be processed, and further ensure integrality of a result of the gene analysis task.

Step 105. The first computing node divides a gene analysis task of the pasting back result sequence set corresponding to the target chromosome region into k gene analysis subtasks, where the k gene analysis subtasks are in a one-to-one correspondence to the k chromosome subregions, and executes in parallel the k gene analysis subtasks.

During specific implementation, after dividing the target chromosome region and the pasting back result sequence set corresponding to the target chromosome region, the first computing node locally divides the gene analysis task of the pasting back result sequence set corresponding to the target chromosome region into k gene analysis subtasks of the pasting back result sequence subsets corresponding to the k chromosome subregions, and executes in parallel the k gene analysis subtasks using a computing resource allocated by the distributed computing system in order to complete a gene analysis task of the pasting back result sequence set corresponding to the target chromosome region. For example, the k gene analysis subtasks are executed in parallel in a contention-based manner and using the computing resource allocated by the distributed computing system to the first computing node.

Step 106. After executing in parallel the k gene analysis subtasks, the first computing node combines results of the k gene analysis subtasks, and uses a combined result as a result of the gene analysis task of the pasting back result sequence set corresponding to the target chromosome region.

Further, the first computing node combines the results of the k gene analysis subtasks to obtain the result of the gene analysis task of the pasting back result sequence set corresponding to the target chromosome region. The result of the gene analysis task is usually a file in a Variant Call Format (VCF) format.

In addition, for the distributed computing system, that the gene analysis task corresponding to the pasting back result sequence set is divided into k gene analysis subtasks, and running of the k gene analysis subtasks are transparent. An architecture of the existing distributed computing system does not need to be changed.

Step 107. The first computing node executes the gene analysis task of the pasting back result sequence set corresponding to the target chromosome region.

Further, if the first computing node determines that the quantity of the pasting back result sequences included in the pasting back result sequence set is less than the pre-determined quantity threshold, the first computing node may determine that the gene analysis task for the pasting back result sequence set is not an uneven task, and may directly execute the gene analysis task using the computing resource allocated by the distributed computing system.

In some feasible implementations, based on that the pre-determined quantity threshold may be the average value of the quantity of the pasting back result sequences included in the pasting back result sequence set corresponding to each chromosome region, and considering that there may be a relatively large quantity of chromosome regions corresponding to which the quantity of the pasting back result sequences included in the pasting back result sequence set is slightly greater than the pre-determined quantity threshold, accordingly, an execution time of the gene analysis task is relatively close to an average execution time. Therefore, a gene analysis task corresponding to such a chromosome region may not be divided again. In this case, when a quantity of pasting back result sequences included in a pasting back result sequence set corresponding to a chromosome region is far greater than the pre-determined quantity threshold, the first computing node considers that a corresponding gene analysis task is an uneven task. For example, when a difference obtained after the pre-determined quantity threshold is subtracted from the quantity of the pasting back result sequences included in the pasting back result sequence set is greater than or equal to a value, or the quantity of the pasting back result sequences included in the pasting back result sequence set is two or more times as much as the pre-determined quantity threshold, the first computing node considers that the corresponding gene analysis task is an uneven task.

The gene analysis task includes one or more of deduplication, local reordering, base quality recalibration, or mutation detection. Likewise, the gene analysis subtask includes one or more of deduplication, local reordering, base quality recalibration, or mutation detection.

In this embodiment of the present disclosure, the computing node distributes the pasting back result sequence corresponding to the to-be-pasted-back DNA read string to the pasting back result sequence set corresponding to the target chromosome region, determines whether the quantity of the pasting back result sequences included in the pasting back result sequence set is greater than or equal to the pre-determined quantity threshold, and if the quantity of the pasting back result sequences included in the pasting back result sequence set is greater than or equal to the pre-determined quantity threshold, divides the pasting back result sequence set into k pasting back result sequence subsets according to the preset division rule, and correspondingly divides the target chromosome region into k chromosome subregions in a one-to-one correspondence to the k pasting back result sequence subsets, and further divides the gene analysis task of the pasting back result sequence set into k gene analysis subtasks, and executes in parallel the k gene analysis subtasks using the computing resource allocated by the distributed computing system, and combines the results of the k gene analysis subtasks and uses a combined result as a result of the gene analysis task corresponding to the target chromosome region. This can improve execution efficiency of the gene analysis task and reduce time overheads of the gene analysis task.

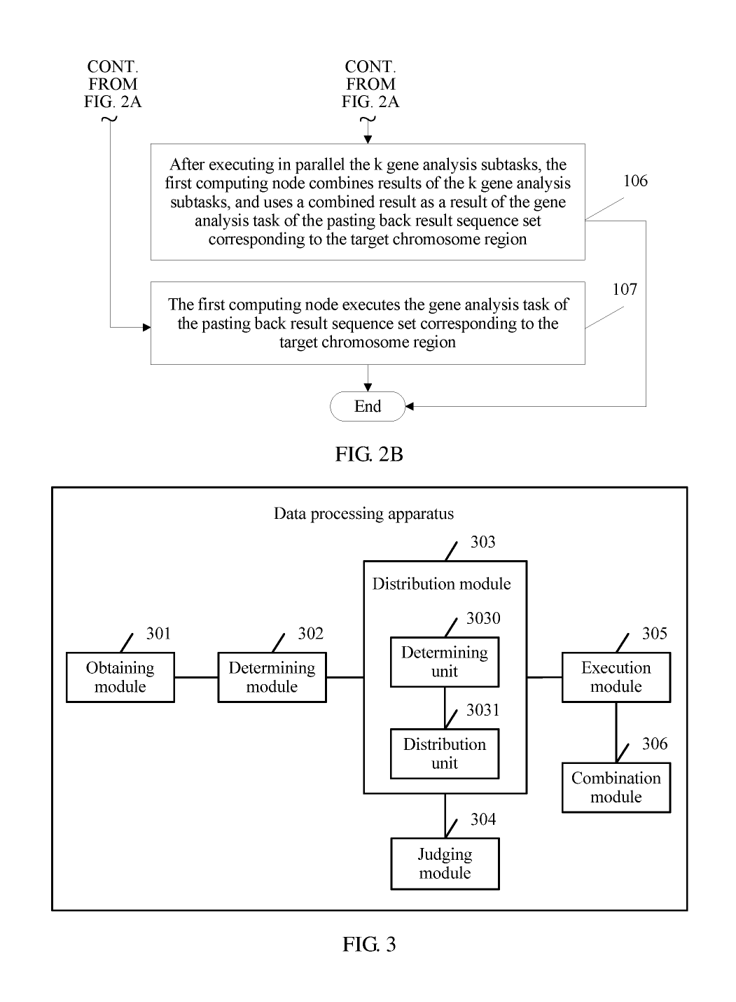

Referring to FIG. 3, FIG. 3 is a schematic structural diagram of a data processing apparatus according to an embodiment of the present disclosure. The data processing apparatus described in this embodiment is applied to a distributed computing system, and the apparatus includes an obtaining module 301 configured to perform a pasting back operation by aligning a to-be-pasted-back DNA read string with a reference gene sequence, and obtain a chromosome location matching the to-be-pasted-back DNA read string, a determining module 302 configured to determine, from a plurality of chromosome regions obtained through division in advance, a target chromosome region in which the chromosome location is located, a distribution module 303 configured to distribute a pasting back result sequence corresponding to the to-be-pasted-back DNA read string to a pasting back result sequence set corresponding to the target chromosome region, a judging module 304 configured to determine whether a quantity of pasting back result sequences included in the pasting back result sequence set is greater than or equal to a pre-determined quantity threshold, where when the judging module 304 determines that the quantity of the pasting back result sequences included in the pasting back result sequence set is greater than or equal to the pre-determined quantity threshold, the distribution module 303 is further configured to divide the pasting back result sequence set into k pasting back result sequence subsets according to a preset division rule, and correspondingly divide the target chromosome region into k chromosome subregions, where the k chromosome subregions are in a one-to-one correspondence to the k pasting back result sequence subsets, and k is an integer greater than or equal to 2, and the distribution module 303 is further configured to divide a gene analysis task of the pasting back result sequence set corresponding to the target chromosome region into k gene analysis subtasks, where the k gene analysis subtasks are in a one-to-one correspondence to the k chromosome subregions, and an execution module 305 configured to execute in parallel the k gene analysis subtasks.

In some feasible implementations, the obtaining module 301 is further configured to obtain a size of a data amount of all to-be-pasted-back DNA read strings.

The determining module 302 is further configured to determine, according to the size of the data amount of all the to-be-pasted-back DNA read strings, a size of a data amount of a pasting back result sequence obtained after all the to-be-pasted-back DNA read strings are pasted back to the reference gene sequence.

The determining module 302 is further configured to determine, according to a quantity of the plurality of chromosome regions obtained through division in advance and the size of the data amount of the pasting back result sequence, a size of an average data amount of a pasting back result sequence set corresponding to each chromosome region.

The determining module 302 is further configured to determine the pre-determined quantity threshold according to the average data amount of the pasting back result sequence set corresponding to each chromosome region and a quantity of pasting back result sequences in a unit of data amount.

In some feasible implementations, the distribution module 303 further includes a determining unit 3030 configured to determine, according to a ratio of the quantity of the pasting back result sequences included in the pasting back result sequence set to the pre-determined quantity threshold, a quantity of the pasting back result sequence subsets k obtained after the pasting back result sequence set is divided, and a distribution unit 3031 configured to divide the pasting back result sequence set into k pasting back result sequence subsets, and divide the target chromosome region into k consecutive chromosome subregions, where the distribution unit 3031 is further configured to correspondingly distribute, according to a chromosome subregion in which a chromosome location corresponding to each pasting back result sequence included in the pasting back result sequence set is located, each pasting back result sequence included in the pasting back result sequence set to the k pasting back result sequence subsets corresponding to the k consecutive chromosome subregions.

In some feasible implementations, when a chromosome location corresponding to a target pasting back result sequence in the pasting back result sequence set is located in two chromosome subregions, the distribution unit 3031 is further configured to distribute the target pasting back result sequence to pasting back result sequence subsets corresponding to both the two chromosome subregions.

In some feasible implementations, the apparatus further includes a combination module 306 configured to combine results of the k gene analysis subtasks, and use a combined result as a result of the gene analysis task of the pasting back result sequence set corresponding to the target chromosome region after the execution module 305 executes in parallel the k gene analysis subtasks.

In some feasible implementations, the gene analysis task includes one or more of deduplication, local reordering, base quality recalibration, or mutation detection.

In this embodiment of the present disclosure, the computing node distributes the pasting back result sequence corresponding to the to-be-pasted-back DNA read string to the pasting back result sequence set corresponding to the target chromosome region, determines whether the quantity of the pasting back result sequences included in the pasting back result sequence set is greater than or equal to the pre-determined quantity threshold, and if the quantity of the pasting back result sequences included in the pasting back result sequence set is greater than or equal to the pre-determined quantity threshold, divides the pasting back result sequence set into k pasting back result sequence subsets according to the preset division rule, and correspondingly divides the target chromosome region into k chromosome subregions in a one-to-one correspondence to the k pasting back result sequence subsets, and further divides the gene analysis task of the pasting back result sequence set into k gene analysis subtasks, and executes in parallel the k gene analysis subtasks using the computing resource allocated by the distributed computing system. This can improve execution efficiency of the gene analysis task and reduce time overheads of the gene analysis task.

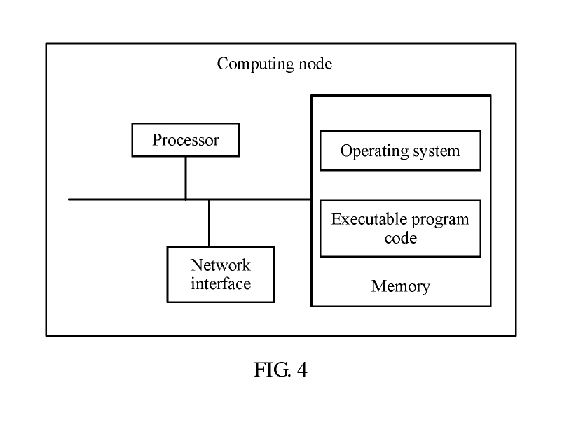

Referring to FIG. 4, FIG. 4 is a schematic structural diagram of a computing node according to an embodiment of the present disclosure. The computing node described in this embodiment is applied to a distributed computing system, and the computing node includes a processor, a network interface, and a memory. The processor, the network interface, and the memory of the computing node may be connected using a bus or in another manner, and bus connection is used as an example in this embodiment of the present disclosure.

The processor (or is referred to as a central processing unit (CPU)) is a computing core and a control core of the computing node. Optionally, the network interface may include a standard wired interface and wireless interface (for example, a WI-FI and a mobile communications interface). The memory is a storage device of the computing node and is configured to store program and data. It may be understood that the memory herein may be a high-speed random access memory (RAM), or may be a non-volatile memory, for example, at least one magnetic disk memory. Optionally, the memory may be at least one storage apparatus that is away from the foregoing processor. The memory provides a storage space. The storage space stores an operating system and executable program code (for example, related service program) of the computing node, including but not limited to a WINDOWS operating system and a LINUX operating system, which is not limited in this the present disclosure.

In this embodiment of the present disclosure, the processor executes, by running the executable program code in the memory, the following operations.

The processor is configured to perform a pasting back operation by aligning a to-be-pasted-back DNA read string with a reference gene sequence, and obtain a chromosome location matching the to-be-pasted-back DNA read string.

The processor is further configured to determine, from a plurality of chromosome regions obtained through division in advance, a target chromosome region in which the chromosome location is located, and distribute a pasting back result sequence corresponding to the to-be-pasted-back DNA read string to a pasting back result sequence set corresponding to the target chromosome region.

The processor is further configured to determine whether a quantity of pasting back result sequences included in the pasting back result sequence set is greater than or equal to a pre-determined quantity threshold, and if the quantity of the pasting back result sequences included in the pasting back result sequence set is greater than or equal to the pre-determined quantity threshold, divide the pasting back result sequence set into k pasting back result sequence subsets according to a preset division rule, and correspondingly divide the target chromosome region into k chromosome subregions, where the k chromosome subregions are in a one-to-one correspondence to the k pasting back result sequence subsets, and k is an integer greater than or equal to 2.

The processor is further configured to divide a gene analysis task of the pasting back result sequence set corresponding to the target chromosome region into k gene analysis subtasks, where the k gene analysis subtasks are in a one-to-one correspondence to the k chromosome subregions, and execute in parallel the k gene analysis subtasks.

In some feasible implementations, before determining whether the quantity of the pasting back result sequences included in the pasting back result sequence set is greater than or equal to the pre-determined quantity threshold, the processor is further configured to obtain a size of a data amount of all to-be-pasted-back DNA read strings.

The processor is further configured to determine, according to the size of the data amount of all the to-be-pasted-back DNA read strings, a size of a data amount of a pasting back result sequence obtained after all the to-be-pasted-back DNA read strings are pasted back to the reference gene sequence.

The processor is further configured to determine, according to a quantity of the plurality of chromosome regions obtained through division in advance and the size of the data amount of the pasting back result sequence, a size of an average data amount of a pasting back result sequence set corresponding to each chromosome region.

The processor is further configured to determine the pre-determined quantity threshold according to the average data amount of the pasting back result sequence set corresponding to each chromosome region and a quantity of pasting back result sequences in a unit of data amount.

In some feasible implementations, a specific manner in which the processor divides the pasting back result sequence set into k pasting back result sequence subsets according to the preset division rule, and correspondingly divides the target chromosome region into k chromosome subregions includes determining, according to a ratio of the quantity of the pasting back result sequences included in the pasting back result sequence set to the pre-determined quantity threshold, a quantity of the pasting back result sequence subsets k obtained after the pasting back result sequence set is divided, dividing the pasting back result sequence set into k pasting back result sequence subsets, and dividing the target chromosome region into k consecutive chromosome subregions, and correspondingly distributing, according to a chromosome subregion in which a chromosome location corresponding to each pasting back result sequence included in the pasting back result sequence set is located, each pasting back result sequence included in the pasting back result sequence set to the k pasting back result sequence subsets corresponding to the k consecutive chromosome subregions.

In some feasible implementations, the processor is further configured to when a chromosome location corresponding to a target pasting back result sequence in the pasting back result sequence set is located in two chromosome subregions, distribute the target pasting back result sequence to pasting back result sequence subsets corresponding to both the two chromosome subregions.

In some feasible implementations, the processor is further configured to after executing in parallel the k gene analysis subtasks, combine results of the k gene analysis subtasks, and use a combined result as a result of the gene analysis task of the pasting back result sequence set corresponding to the target chromosome region.

In some feasible implementations, the gene analysis task includes one or more of deduplication, local reordering, base quality recalibration, or mutation detection.

In this embodiment of the present disclosure, the computing node distributes the pasting back result sequence corresponding to the to-be-pasted-back DNA read string to the pasting back result sequence set corresponding to the target chromosome region, determines whether the quantity of the pasting back result sequences included in the pasting back result sequence set is greater than or equal to the pre-determined quantity threshold, and if the quantity of the pasting back result sequences included in the pasting back result sequence set is greater than or equal to the pre-determined quantity threshold, divides the pasting back result sequence set into k pasting back result sequence subsets according to the preset division rule, and correspondingly divides the target chromosome region into k chromosome subregions in a one-to-one correspondence to the k pasting back result sequence subsets, and further divides the gene analysis task of the pasting back result sequence set into k gene analysis subtasks, and executes in parallel the k gene analysis subtasks using the computing resource allocated by the distributed computing system. This can improve execution efficiency of the gene analysis task and reduce time overheads of the gene analysis task.

It should be noted that, for brief description, the foregoing method embodiments are represented as a series of actions. However, a person skilled in the art should appreciate that the present disclosure is not limited to the described order of the actions, because according to the present disclosure, some steps may be performed in other orders or simultaneously. In addition, a person skilled in the art should also appreciate that all the embodiments described in the specification are example embodiments, and the related actions and modules are not necessarily mandatory to the present disclosure.

A person of ordinary skill in the art may understand that all or some of the steps of the methods in the embodiments may be implemented by a program instructing relevant hardware. The program may be stored in a computer readable storage medium. The storage medium may include a flash memory, a read-only memory (ROM), a RAM, a magnetic disk, and an optical disc.

The foregoing has described in detail the data processing method and apparatus, and the computing node provided in the embodiments of the present disclosure. In this specification, specific examples are used to describe the principle and implementations of the present disclosure, and the description of the embodiments is only intended to help understand the method and core idea of the present disclosure. In addition, a person of ordinary skill in the art may, based on the idea of the present disclosure, make modifications with respect to the specific implementations and the application scope. Therefore, the content of this specification shall not be construed as a limitation to the present disclosure.

What is claimed is:

1. A data processing method, the data processing method being applied to a distributed computing system, and the data processing method comprising:
performing, by a first computing node, a pasting back operation by aligning a to-be-pasted-back deoxyribonucleic acid (DNA) read string with a reference gene sequence, the distributed computing system comprising a plurality of computing nodes and the first computing node being any one of the computing nodes;
obtaining, by the first computing node, a chromosome location matching the to-be-pasted-back DNA read string;
searching, by the first computing node from a plurality of chromosome regions obtained through division in advance, a target chromosome region in which the chromosome location is located;
distributing, by the first computing node, a pasting back result sequence corresponding to the to-be-pasted-back DNA read string to a pasting back result sequence set corresponding to the target chromosome region;
dividing, by the first computing node, the pasting back result sequence set into k pasting back result sequence subsets according to a preset division rule and the target chromosome region into k chromosome subregions when the quantity of the pasting back result sequences comprised in the pasting back result sequence set is greater than or equal to the pre-determined quantity threshold, the k chromosome subregions being in a one-to-one correspondence to the k pasting back result sequence subsets, and the k being an integer greater than or equal to two;
dividing, by the first computing node, a gene analysis task of the pasting back result sequence set corresponding to the target chromosome region into k gene analysis subtasks, the k gene analysis subtasks being in a one-to-one correspondence to the k chromosome subregions; and
executing in parallel, by the first computing node, the k gene analysis subtasks.

2. The data processing method of claim 1, wherein before determining whether the quantity of the pasting back result sequences comprised in the pasting back result sequence set is greater than or equal to the pre-determined quantity threshold, the method further comprises:
obtaining, by the first computing node, a size of a data amount of all to-be-pasted-back DNA read strings;
determining, by the first computing node according to the size of the data amount of all the to-be-pasted-back DNA read strings, a size of a data amount of a pasting back result sequence obtained after all the to-be-pasted-back DNA read strings are pasted back to the reference gene sequence;
determining, by the first computing node according to a quantity of the chromosome regions obtained through division in advance and the size of the data amount of the pasting back result sequence, a size of an average data amount of a pasting back result sequence set corresponding to each chromosome region; and
determining, by the first computing node, the pre-determined quantity threshold according to the average data amount of the pasting back result sequence set corresponding to each chromosome region and a quantity of pasting back result sequences in a unit of data amount.

3. The data processing method of claim 1, wherein dividing the pasting back result sequence set into k pasting back result sequence subsets and the target chromosome region into the k chromosome subregions comprises:
determining, by the first computing node according to a ratio of the quantity of the pasting back result sequences comprised in the pasting back result sequence set to the pre-determined quantity threshold, a quantity of the pasting back result sequence subsets obtained after the pasting back result sequence set is divided, the quantity of the pasting back result sequence subsets being equal to the k;
dividing, by the first computing node, the pasting back result sequence set into the k pasting back result sequence subsets;
dividing, by the first computing node, the target chromosome region into k consecutive chromosome subregions; and
distributing, by the first computing node according to a chromosome subregion in which a chromosome location corresponding to each pasting back result sequence comprised in the pasting back result sequence set is located, each pasting back result sequence comprised in the pasting back result sequence set to the k pasting back result sequence subsets corresponding to the k consecutive chromosome subregions.

4. The data processing method of claim 3, wherein a chromosome location corresponding to a target pasting back result sequence in the pasting back result sequence set is located in two chromosome subregions, and the data processing method further comprising distributing, by the first computing node, the target pasting back result sequence to pasting back result sequence subsets corresponding to both the two chromosome subregions.

5. The data processing method of claim 1, wherein after executing in parallel the k gene analysis subtasks, the data processing method further comprises:
combining, by the first computing node, results of the k gene analysis subtasks; and
setting, by the first computing node, a combined result as a result of the gene analysis task of the pasting back result sequence set corresponding to the target chromosome region.

6. The data processing method of claim 5, wherein the result of the gene analysis task is in a Variant Call Format (VCF).

7. The data processing method of claim 1, wherein the gene analysis task comprises deduplication.

8. The data processing method of claim 1, wherein the gene analysis task comprises local reordering.

9. The data processing method of claim 1, wherein the gene analysis task comprises base quality recalibration.

10. The data processing method of claim 1, wherein the gene analysis task comprises mutation detection.

11. A data processing apparatus, comprising:
a non-transitory computer-readable storage medium storing programming instructions; and
at least one processor coupled to the non-transitory computer-readable storage medium, the programming instructions causing the at least one processor to:
perform a pasting back operation by aligning a to-be-pasted-back deoxyribonucleic acid (DNA) read string with a reference gene sequence;
obtain a chromosome location matching the to-be-pasted-back DNA read string;
determine, from a plurality of chromosome regions obtained through division in advance, a target chromosome region in which the chromosome location is located;
distribute a pasting back result sequence corresponding to the to-be-pasted-back DNA read string to a pasting back result sequence set corresponding to the target chromosome region;
determine whether a quantity of pasting back result sequences comprised in the pasting back result sequence set is greater than or equal to a pre-determined quantity threshold;
divide the pasting back result sequence set into k pasting back result sequence subsets according to a preset division rule and the target chromosome region into k chromosome subregions when the quantity of the pasting back result sequences comprised in the pasting back result sequence set is greater than or equal to the pre-determined quantity threshold, the k chromosome subregions being in a one-to-one correspondence to the k pasting back result sequence subsets, and the k being an integer greater than or equal to two;
divide a gene analysis task of the pasting back result sequence set corresponding to the target chromosome region into k gene analysis subtasks, the k gene analysis subtasks being in a one-to-one correspondence to the k chromosome subregions; and
execute in parallel the k gene analysis subtasks.

12. The data processing apparatus of claim 11, wherein the programming instructions further cause the at least one processor to:
obtain a size of a data amount of all to-be-pasted-back DNA read strings;
determine, according to the size of the data amount of all the to-be-pasted-back DNA read strings, a size of a data amount of a pasting back result sequence obtained after all the to-be-pasted-back DNA read strings are pasted back to the reference gene sequence;
determine, according to a quantity of the chromosome regions obtained through division in advance and the size of the data amount of the pasting back result sequence, a size of an average data amount of a pasting back result sequence set corresponding to each chromosome region; and
determine the pre-determined quantity threshold according to the average data amount of the pasting back result sequence set corresponding to each chromosome region and a quantity of pasting back result sequences in a unit of data amount.

13. The data processing apparatus of claim 11, wherein the programming instructions further cause the at least one processor to:
determine, according to a ratio of the quantity of the pasting back result sequences comprised in the pasting back result sequence set to the pre-determined quantity threshold, a quantity of the pasting back result sequence subsets obtained after the pasting back result sequence set is divided, the quantity of the pasting back result sequence subsets being equal to the k;
divide the pasting back result sequence set into the k pasting back result sequence subsets;
divide the target chromosome region into k consecutive chromosome subregions;
distribute, according to a chromosome subregion in which a chromosome location corresponding to each pasting back result sequence comprised in the pasting back result sequence set is located, each pasting back result sequence comprised in the pasting back result sequence set to the k pasting back result sequence subsets corresponding to the k consecutive chromosome subregions.

14. The data processing apparatus of claim 13, wherein a chromosome location corresponding to a target pasting back result sequence in the pasting back result sequence set is located in two chromosome subregions, and the programming instructions further causing the at least one processor to distribute the target pasting back result sequence to pasting back result sequence subsets corresponding to both the two chromosome subregions.

15. The data processing apparatus of claim 11, wherein after executing in parallel the k gene analysis subtasks, the programming instructions further cause the at least one processor to:
combine results of the k gene analysis subtasks; and
set a combined result as a result of the gene analysis task of the pasting back result sequence set corresponding to the target chromosome region.

16. The data processing apparatus of claim 15, wherein the result of the gene analysis task is in a Variant Call Format (VCF).

17. The data processing apparatus of claim 11, wherein the gene analysis task comprises deduplication.

18. The data processing apparatus of claim 10, wherein the gene analysis task comprises local reordering.

19. The data processing apparatus of claim 10, wherein the gene analysis task comprises base quality recalibration.

20. The data processing apparatus of claim 10, wherein the gene analysis task comprises mutation detection.
